(19) United States
(12) Patent Application Publication
(10) Pub. No.: US 2026/0364239 A1
(43) Pub. Date: Nov. 19, 2026

(54) APHERESIS OF WHOLE BLOOD

(71) Applicant: Isaac ELIAZ, Santa Rosa, CA (US)

(72) Inventor: ISAAC ELIAZ, Santa Rosa, CA (US)

(21) Appl. No.: 19/397,920

(22) Filed: Nov. 22, 2025

Related U.S. Application Data
(63) Continuation of application No. 17/964,644, filed on Oct. 12, 2022.
(60) Provisional application No. 63/256,567, filed on Oct. 16, 2021.

(51) Int. Cl. A61M 1/34

(52) U.S. Cl. CPC A61M 1/3496

(57) ABSTRACT

A method for performing apheresis of mammals, including humans, is set forth which does not require separation of the blood into plasma or any other portion. Termed whole blood apheresis herein, this advance makes it possible to perform apheresis more quickly and efficiently with less stress for the patient. This application also discloses important advances in apheresis for therapeutic treatments, including treatments for sepsis and AKI using whole blood apheresis, and immunotherapy where targets that interfere with recovery are removed by apheresis and gene-engineered fragments previously removed are reintroduced. Use of selective withdrawal through apheresis expands possible resolutions of illnesses and conditions previously thought to be untreatable.

### PRIORITY DATA AND INCORPORATION BY REFERENCE

This application is a Utility U.S. Patent Application which claims priority from U.S. Provisional Application 63/256,567 filed Oct. 16, 2021. While no other claim to priority is made, this case is related to a family of cases directed to the treatment of mammals, including humans, relying in part or in whole on the technique if apheresis. Related cases include those directed to apheresis relying on selective withdrawal of a target such as galectin-3, as disclosed in U.S. Pat. No. 8,764,695. This application is also related to U.S. Pat. No. 10,953,148 which is directed to an apparatus for performing that sort of apheresis. The subject matter of this application is also related to patents such as U.S. Pat. No. 11,389,476 directed to a method of treating mammals for sepsis using apheresis.

### BACKGROUND OF THE INVENTION

### Field of the Invention

As suggested above, this application is directed to the treatment of mammalian patients using apheresis which may comprise the use of selective withdrawal of target compounds such as galectin-3. Selective withdrawal refers to the use of targeted binding agents, such as antibodies, chemical binders like modified citrus pectin, or natural ligands like TNFα, and inhibitors like PDL-1/2 inhibitors, that can be presented in a column, filter or other passageway of an apheresis device such that blood flowing through the device is exposed to the binding agent which selectively withdraws from the blood the target, which may be a protein like Galectin-3 or a protein which, for instance, interferes with the body's mechanisms to deal with immune threats, such as TNFα or PDL-1/2. This application details a strategy for apheresis using whole blood, rather than requiring separation of blood plasma as opposed to other blood components such as blood cells and platelets. This substantially simplifies the process, making it easier to tolerate, less expensive and more broadly applicable to individual patients and procedures. This application also addresses the opportunities for immune therapy using apheresis (of whole blood or plasma only) opened up, in part, by these new advances.

### SUMMARY OF THE INVENTION

This invention discloses and presents actual treatment of blood of mammalian patients using apheresis but treating the blood without separation or pretreatment into fractions like platelets, plasma, whole cells and the like.

This dramatically simplifies the apheresis process, making it less difficult and cumbersome for the patient. By conducting apheresis without separation of blood fragments, or otherwise conditioning the mammal prior to apheresis. This makes the practice of apheresis dramatically less expensive and less time consuming. It also reduces stress and difficulties encountered in prior art practice, without loss of effectiveness, particularly in the environment of apheresis practiced with selective withdrawal of a target such as galectin-3 or other blood component. In this application, apheresis practiced on whole blood is referred to as just that-whole blood apheresis. This is distinguished from prior art processes where blood is diverted from the body and then separated into components, plasma, white blood cells, platelet fractions, etc. In “whole blood apheresis” as the term is used herein, blood is diverted from the body, but introduced directly to the apheresis device where elements may be withdrawn from that blood, and other elements may be introduced to the patient's blood before it is returned to the body.

### DETAILED DESCRIPTION OF THE INVENTION

Whole blood apheresis was demonstrated as effective in the course of a study evaluating the efficacy of rats with sepsis induced by Cecal Ligation and Puncture Induced Sepsis (CPL), a well-established model. CPL in rodents is considered the gold standard in sepsis research and the most widely used model for experimental sepsis. Developed more than thirty (30) years ago, CLP is considered a realistic model for the induction of polymicrobial sepsis for studying the underlying mechanism. CLP features ligation below the ileocecal valve, the sphincter muscle at the junction of the ileum (last portion of the small intestine) and the colon (first portion of the large intestine), after midline laparotomy (an incision is made down the middle of the abdomen to gain access), followed by needle puncture of the cecum. As the cecum is an endogenous source of bacterial contamination, perforation of the cecum results in bacterial peritonitis, which is followed by translocation of mixed enteric bacteria into the blood system. At the onset of sepsis, bacteremia then triggers systemic activation of the inflammatory response, subsequent septic shock, multiorgan dysfunction, and death. When the CLP model is used in rodents, they show disease patterns with typical symptoms of sepsis or septic shock, such as hypothermia, tachycardia, and tachypnea.

Sepsis is the leading cause of mortality in intensive care units (ICU) worldwide and is the most common cause of acute kidney injury (AKI) in the modern era. Across resource-rich and resource-limited settings, sepsis and sepsis-associated acute kidney injury (S-AKI) are associated with significant morbidity and mortality, as well as high healthcare costs. Annual sepsis incidence in the United States of America (USA) is greater than 1.7 million and responsible for one in three hospital deaths. Further, S-AKI is disproportionately responsible for sepsis mortality and severe morbidity, accounting for over half of sepsis-related deaths. In S-AKI survivors, impaired kidney function increases the risk of chronic kidney disease (CKD) and remains a significant factor affecting long-term disability, quality of life, and survival.

Sepsis is a potentially fatal complex immune disorder resulting from the disregulation of multiple host defense pathways in response to infection. Sepsis is characterized by the extensive release of cytokines, among other inflammatory mediators, which leads to fatal organ damage. In the USA, the incidence of sepsis and S-AKI remain high, with a dramatic rise in AKI incidence from 7.2% in 2002 to 20% in 2012 among patients hospitalized at tertiary care hospitals. 5 Current management of S-AKI is limited to antimicrobial therapies and organ support, including the provision of hemodialysis or continuous renal replacement therapy.

There are no approved therapies to prevent, interrupt the evolution, or hasten recovery after S-AKI. Novel therapeutic interventions remain an unmet and critical need in the management of sepsis and S-AKI.

Galectin-3(Gal-3 ) is a soluble 32-35 kilodalton (kDa) member of the lectin family of proteins. Gal-3 is expressed in most human tissues, including an array of immune cells (such as macrophages, dendritic cells, eosinophils, mast cells, natural killer cells, activated T-cells, and activated B-cells), epithelial cells, endothelial cells, and sensory neurons. The scientific literature identifies Gal-3 as a driver in pro-inflammatory and profibrotic signaling in a wide range of acute and chronic diseases; including sepsis, AKI, CKD, heart failure, non-alcoholic steatohepatitis (NASH), idiopathic pulmonary fibrosis (IPF), and autoimmune disease, as well as an oncoprotein in tumorigenesis. In response to infectious and toxic insults, Gal-3 functions as an “alarmin,” instigating an immune response. Gal-3 is upregulated, brought to the cell surface, and secreted into the circulation. Gal-3 activates membrane toll-like receptors, ignites intracellular inflammasome protein complexes and leads to cytokine release, hyper-inflammation, and immune dysregulation.

Notably, inflammasome activity has been shown to contribute to pulmonary inflammation and acute respiratory distress syndrome and leads to both higher mortality and reduced microbial clearance in the setting of Coronavirus Disease 2019(COVID-19 ), influenza, and bacterial superinfection. Additionally, by forming ligand-Gal-3 complexes, cell surface lattice structures, and binding of bioactive glycoproteins and glycolipids, Gal-3 fuels excessive inflammation and fibrosis, which contribute to renal dysfunction and failure.

Multiple studies from our group and others show that Gal-3 is not just a biomarker but plays an orchestrating causal role in the pathogenesis of sepsis and S-AKI. In a murine model of sepsis secondary to pulmonary infection, Gal-3 was upregulated and secreted into the extracellular space and circulation in the septic mice. Elevated serum Gal-3 concentrations were associated with a hyperinflammatory response, cellular death, and increased vascular injury. Gal-3 knockout (KO) mice demonstrated reduced lung pathology and significantly improved survival compared to wild-type mice (p=0.0003). Further, Gal-3 KO mice exhibited reduced inflammation and tissue damage, as well as significantly lower levels of inflammatory markers, inflammatory mediators, and markers of vascular injury such as C-reactive protein (CRP), interleukin (IL)-1β, IL-6, tumor necrosis factor-α (TNF), thrombopoietin, and fibrinogen.

We have demonstrated the role of Gal-3 in sepsis and S-AKI through both oral Gal-3 inhibition and removal of Gal-3 by apheresis in rat models. In the recent study published in Critical Care, we examined 7-day mortality, serum Gal-3, IL-6, and creatinine concentrations in a rat cecal ligation and puncture (CLP) model of sepsis and S-AKI.27 Both serum Gal-3 and IL-6 were elevated significantly following CLP. Rats pre-treated with an oral Gal-3 inhibitor at 400 mg/kg/d and 1200 mg/kg/d prior to the CLP procedure had significantly reduced serum concentrations of both Gal-3 and IL-6 compared to controls. Notably, circulating Gal-3 levels consistently increased and spiked earlier than IL-6, showing its role as an upstream mediator in the inflammatory cascade in sepsis and S-AKI. Seven-day mortality was significantly lower in the Gal-3 inhibitor 400 mg (28%, p=0.03) and 1200 mg (22%, p=0.001) groups, compared to controls (61%). Additionally, AKI incidence was significantly reduced from 89% in the control group to 44% (p=0.007) in both Gal-3 inhibitor groups based on RIFLE (Risk of renal dysfunction, Injury to kidney, Failure or Loss of kidney function, and End-stage kidney disease) criteria. The oral Gal-3 inhibitor used in these studies was Pectasol® modified citrus pectin (P-MCP), a low molecular weight pectin that directly inhibits Gal-3 by binding to its carbohydrate recognition domain. The PI developed Pectasol® as a dietary supplement, and as a pectin, it is classified as generally regarded as safe (GRAS) by the FDA. The effect of P-MCP has been confirmed in multiple conditions and animal models. In the companion study evaluating patients with sepsis, serum Gal-3 on admission to the ICU was an independent predictor of ICU mortality (p=0.04) and AKI (p=0.01). We recently were able to perform rat Gal-3 depletion apheresis in the CLP model. We demonstrated a significant difference in survival between the Gal-3 apheresis group (survival: 9/10), and the sham apheresis group (survival: 1/9) (p<0.01). We discuss this study in greater detail in the milestone section below.

In a study of ischemia/reperfusion (I/R) injury using a renal pedicle occlusion murine model, Gal-3 KO mice showed a significant reduction in acute tubular necrosis compared to controls (p<0.0001) and enhanced tubular regeneration (p<0.005). Further, Gal-3 KO mice exhibited significantly lower levels of IL-6 (p<0.05) and IL-1β (p<0.05), as well as reduced reactive oxygen species (p=0.003). In our most recent rat model study of Gal-3 in I/R injury, Gal-3 and IL-6 were significantly elevated from baseline following renal pedicle occlusion, with Gal-3 levels rising prior to IL-6.36 Pre-treatment with a Gal-3 inhibitor resulted in significantly reduced serum Gal-3 and IL-6, renal tubular injury, and apoptosis, as well as improved kidney function (p<0.05). In the companion study of 52 patients admitted to the ICU following coronary artery bypass graft (CABG) without pre-existing kidney disease, the serum Gal-3 concentration on ICU admission was an independent predictor of AKI and performed better as an early biomarker of AKI than neutrophil gelatinase-associated lipocalin (NGAL), Cystatin C (CysC), and serum creatinine (Cr) (Area Under the Receiver Operating Characteristic Curve [AUC-ROC]: Gal-3 0.890; NGAL 0.763; Cr 0.773). It is important to note that in human studies, serum Gal-3 elevations persist for longer durations. For example, in an observational study of 645 ICU patients with incident AKI, serum Gal-3 levels remained elevated at hospital discharge with the level of Gal-3 correlating with severity of AKI.

Gal-3 inhibition has been demonstrated to reduce inflammation and prevent renal fibrosis in multiple murine models of AKI. In a murine study utilizing a folic acid-induced kidney injury model, mice were treated with an oral Gal-3 inhibitor starting one week before folic acid injection. The Gal-3 inhibitor group demonstrated a significant reduction in acute gross kidney swelling. The pre-treated mice demonstrated a 30% reduction in Gal-3 protein expression at two weeks following folic acid injection. Pre-treatment with a Gal-3 inhibitor significantly decreased renal fibrosis (p<0.05), as well as significantly reduced levels of fibrotic markers (collagen I, fibronectin, and transforming growth factor-beta [p<0.05]), pro-inflammatory cytokines (IL-1b [p<0.05] and TNF-α [p<0.05]), and apoptosis (p<0.01).30 In other studies, Gal-3 inhibitors have successfully reduced inflammation and fibrosis in multiple organ injury and disease models. Notably, in patients with impaired kidney function, elevated serum Gal-3 is associated with rapid deterioration of kidney function, incident CKD, and all-cause mortality.

The depletion of Gal-3 in a sepsis model is unprecedented, differentiating our approach from endotoxin removal and other extracorporeal strategies. Potential future applications include other etiologies of AKI, CKD, NASH, and in enhancing immunotherapies in cancer, heart failure, myocardial infarction, and IPF.

Finally, several groups have published studies that show that elevated serum concentrations of Gal-3 predict progression to severe COVID-19 in patients infected with SARS-COV-261,62 and suggest that Gal-3 is an attractive upstream target to regulate inflammatory response and prevent cytokine storm syndrome in these patients. Thus, while our focus is on sepsis/AKI, the potential for Gal-3 depletion therapy to treat acute COVID-19 provides additional urgency to our application.

In summary, multiple studies demonstrate the orchestrating role of Gal-3 in the pathogenesis of sepsis and AKI using multiple methodologies, including oral pharmacological inhibitors and KO mice, as well as observational human data. These studies are consistent with the critical role of Gal-3 in accentuating the inflammatory and fibrotic responses to acute injury. Given the evolving evidence consistent with a causal role of Gal-3 in sepsis and S-AKI, and the urgent need for therapeutic interventions, we have proposed Gal-3 specific apheresis as a novel treatment for sepsis and S-AKI. We postulate that the rapid and efficient depletion of excess plasma Gal-3 will inhibit and potentially reverse the immune dysregulation underlying sepsis, reducing both sepsis and S-AKI morbidity and mortality. The proposed project addresses the urgent need for a practical, rapidly acting therapeutic intervention that may be performed in patients with sepsis and S-AKI.

We disclose here a novel treatment for sepsis and S-AKI through depletion of serum Gal-3 using our proprietary Gal-3 selective apheresis column, XGal3®. Our proposal includes multiple innovative components. This unique medical device integrates a first-of-its-kind selective Gal-3 adsorption capture molecule into an apheresis column. Gal-3 depletion apheresis is a novel product and procedure invented by the PI, Dr. Eliaz, who developed the first commercially available Gal-3 inhibitor and has been involved in Gal-3 research and clinical application for over 25 years. Over the past six years, in collaboration with leading experts worldwide, our team has developed a proprietary monoclonal Gal-3 capture antibody that selectively binds to Gal-3. The Gal-3 apheresis column is compatible with clinical apheresis systems currently used in hospitals and clinics, simplifying regulatory and commercialization pathways. The XGal3® filter is compatible with pharmaceutical treatments, as well as with added/other extracorporeal therapies.

Gal-3 specific therapeutic apheresis has the potential to reduce morbidity and mortality associated with sepsis and S-AKI—a condition for which there is no effective treatment. In addition, our novel approach also has the potential to mitigate deterioration in kidney function and prevent or improve CKD in sepsis survivors.

Therapeutic apheresis offers an effective and safe therapeutic option compared to drug treatments. Pharmacological interventions have limits due to pharmacokinetics, drug-drug interactions, toxicities, and other adverse effects. These limitations become increasingly more complex in critically ill patients. The Gal-3 selective apheresis column, XGal3®, offers the potential to rapidly and safely remove Gal-3 from the circulation without the toxicities, side effects, and dose limitations. In addition, Gal-3 specific apheresis can be performed repeatedly and as often as necessary. Of note, Gal-3 regenerates quickly at the cellular/tissue level, and depletion, inhibition, and KO of Gal-3 have not shown any harm in animal models or humans.

Gal-3 functions by generating pentamer complexes that cross-link with target ligands. All developed Gal-3 inhibitors function as competitive inhibitors at the carbohydrate recognition domain (CRD) and therefore are limited to blocking Gal-3. In contrast, XGal3® antibodies bind the Gal-3 pentamer at the N-terminal, allowing it to remove Gal-3 monomers and pentamers with their associated pathogenic ligands from the circulation. Oral Gal-3 inhibitors are in development, but none are being tested for sepsis and S-AKI indications. GS-100-a form of modified citrus pectin developed by La Jolla Pharmaceuticals-was initially targeted to treat CKD but was discontinued for financial reasons. Unlike the rapid, efficient removal offered by XGal3®, pharmacological inhibitor efficacy is contingent on potency, specificity, metabolism, the strength of Gal-3-ligand interactions, and side effects profile. Additionally, Gal-3 inhibitors are subject to competition with endogenous bound CRD ligands and may lead to off-target effects by binding to other galectins. In contrast, the design of the XGal3® column enables selective and rapid removal of plasma Gal-3 without competition for ligand binding, drug-related complications, or off-target effects.

Extracorporeal procedures for sepsis have included therapeutic plasma exchange (TPE) and filtering columns. In a 2014 meta-analysis of four randomized controlled trials (RCT), TPE exhibited no association with overall mortality.

Approval in Europe of the Cytosorb® (CytoSorbents Europe GmbH, Berlin, Germany) apheresis column to remove IL-6, IL-10, and TNF has proceeded, but with limited success. Polymyxin B cartridge, an extracorporeal hemoperfusion device (PMX-DHP. Toray Medical Co., Tokyo, Japan), is a therapy in Japan and Western Europe for endotoxin removal. During the COVID-19 pandemic, both Cytosorb® columns and the Polymyxin B device received FDA Emergency Authorization in the US for use in critically ill COVID-19 patients. However, neither therapy has demonstrated a significant effect on survival thus far. Other extracorporeal strategies have included high-volume hemofiltration, hemoadsorption, coupled plasma filtration adsorption, high cutoff membranes, and hemoperfusion. Continuous hemodiafiltration using a polymethylmethacrylate (PMMA) membrane hemofilter (PMMA-CHDF, Toray Medical Co., Tokyo, Japan) to remove multiple pro-and anti-inflammatory cytokines has shown conflicting and limited results for the treatment of sepsis in clinical research. In a meta-analysis of RCTs using hemoperfusion with polymyxin B, the authors found no effect on 28-day mortality. These developments demonstrate the urgent need for effective therapies for the treatment of sepsis and the growing interest in apheresis as a therapeutic approach for sepsis. Though many others have tried and failed to develop effective apheresis-based therapies for sepsis, they have all relied on non-specific absorption or clearance of a wide array of pro-and anti-inflammatory mediators. Our approach is fundamentally different in that we target an upstream mediator of the inflammatory response (Gal-3), a novel target for apheresis that we believe will prove more effective. Our approach and specific IP allows us to combine Gal-3 depletion with other apheresis and filtration columns and devices, if required. For example:

Gal-3 depletion can be combined with renal replacement therapy (RRT) in S-AKI patients in the ICU.

We have completed significant milestones: demonstrated Gal-3 depletion from serum with an antibody (Ab); published a proof-of-concept (POC) study in a porcine cutaneous inflammatory injury model; developed a proprietary anti-Gal-3 Ab with successful immobilization; and developed an apheresis column that efficiently removes Gal-3. We established the time course of changes in serum Gal-3 and serum IL-6 concentrations in a septic rat model of circulation; performed therapeutic apheresis in healthy rats; showed that inhibition of Gal-3 effectively reduces serum Gal-3 and systemic inflammation, protects against S-AKI and enhances survival in sepsis in rat models; successfully completed a POC study with a rat CLP model for sepsis and S-AKI that showed that removing Gal-3 from the circulation dramatically reduced mortality; and developed the prototype Gal-3 selective apheresis column for human clinical use.

### SPECIFIC EXAMPLE OF WHOLE BLOOD APHERESIS

We screened commercially available anti-rat Gal-3 antibodies, but none of them performed well enough. We developed a high-affinity anti-rat Gal-3 Ab de novo, using rabbits and rat Gal-3 antigen, and assessed the top eight positive clones from concentration-adjusted ELISA plates coated with recombinant rat Gal-3 to estimate affinity. Evaluation of top clones was then performed using surface plasmon resonance (SPS). The equilibrium dissociation constant (KD) for the highest affinity clone was 2.889E-10, which is more than sufficient After we developed the new anti-rat Gal-3 Ab, we successfully coupled it to sepharose beads and created 0.4ml mini columns of the activated resin and sham mini columns.

We then attempted to perform the key efficacy study to evaluate the impact of Gal-3 apheresis on the survival of rats that had undergone CLP. Unfortunately, the prolonged apheresis procedure and the plasma separation which slowed down the flow rate performed, just one hour after the CLP procedure, was too harsh for the rats, and all animals in both the sham and active group did not survive the procedure.

We therefore performed whole blood apheresis/filtration, using the same mini column. As a result, we were finally able to complete the originally-proposed Gal-3 apheresis depletion study with 19 rats (10 using active Gal-3 depletion columns and 9 using sham empty columns) with apheresis performed 1 hour post CLP for 90 minutes.

The mini columns used were packed with 0.4 ml activated Sepharose with 2 mg/ml of our anti rat gal-3 antibody. Flow rate was 0.5-0.8 ml/minute.

Nine of the 10 treated rats survived to the pre-specified 7-day endpoint, compared to only 1 out of 9 of the rats in the control group that received the sham treatment survived. (All surviving animals were euthanized at seven days in accordance with the protocol.) This new result is a dramatic and significant (p<0.001) demonstration that Gal-3 apheresis is effective in attenuating sepsis. An ex vivo study was performed to confirm the ability of the anti-Gal-3 (rat) antibody to deplete Gal-3. An additional rat was subjected to renal ischemia-reperfusion injury (I/R), plasma was collected 2 hours after reperfusion, and ex vivo depletion was performed in an active column. The ex vivo study confirmed that the active columns depleted Gal-3 levels (79% vs. 2% for a sham column). It is important to note that our anti-Gal-3 (rat) antibody is less effective than our anti-Gal-3 (human) antibody (>90%). We therefore expect the treatment to translate well to humans. Humans will better tolerate the apheresis procedure, and can receive supplementary fluids as needed.

### IMMUNOTHERAPY OPPORTUNITIES

Among the many applications that apheresis lends itself to, and which may be improved in both effectiveness and ease through whole blood apheresis, is immunotherapy. Existing treatments and techniques have been widely discussed, and include PD-1 inhibitors and the like, tumor infiltrating lymphocyte (TILs) treatment, CAR-T cells, induction and return of stem cell infusion, and similar, generally targeting various forms of cancer. All of these therapies can be improved using apheresis. Currently, much focus is on the use of PD-1 and PDL-1 inhibitors to permit cancer treatment to be effective. Apheresis makes it possible, using the techniques described herein and which may include whole blood apheresis or apheresis with plasma separation, to enhance these treatments in a dramatic way.

Thus, rather than relying simply on the administration of agents that inhibit PD-1 and PDL-1 (inhibitors) one can now pass the blood through the apheresis device or column, withdraw the PD-1 and PDL-1 agents from the blood by passing them through antibodies (or other ligands) in the apheresis column specific for PD-1, and then return the blood to the patient such that the interference presented by PD-1 is reduced. The treatment may be augmented by administration of inhibitors, introduced to the blood before its return to the patient, or preferably after the conclusion of the apheresis procedure, and ideally as close to it as possible. TIL treatment, CAR-T cell immunotherapy and induction and return of stem cells all call for the removal of target cells or agents from the patient. Often the targets are then modified genetically, and then reintroduced to the body. This procedure can be simplified and enhanced by the use of apheresis-both for the collection of the agent such as a stem cells and T-Cells for CAR-T immunotherapy, TIL and the like and for administration. In these methods, the collected agents are harvested, and modified, genetically. They must then be returned to the patient. All of these methods may be practiced using apheresis, either whole blood or plasma separation-based apheresis, making the procedure faster, easier and more effective, in that selective withdrawal may be combined with administration of additional agents, to heighten effectiveness. For example, Soluble PD-L1 with PD-1-binding capacity exists in the plasma of patients with cancer, for example non-small cell lung cancer. PD-L1 is one of the important immune checkpoint molecules that can be targeted by cancer immunotherapies. PD-L1 has a soluble form (sPD-L1) and a membrane-bound form (mPD-L1). When we remove the soluble PD-L1 (sPD-L1) due to gradient equilibrium, we can expect the mPD-L1 to be released into the blood, as well as reduce the expression of mPD-L1, thus increasing the presence of sPD-L1. In this way apheresis of whole blood or plasma can not only deplete the sPD-L1, but also the membranous mPD-L1. This will allow for better response to the different PD-L1 inhibitors and can also allow for reduced dose with less toxicity. The concurrent or serial removal of related compounds such as galectin-3, inflammatory cytokines such as IL1B, IL-6, IL-4, IL-8, TNF Alpha, NF Kappa Beta, and others can further enhance the efficacy of immunotherapy while addressing its inflammatory based toxicity. Similar approaches can be utilized pre or post dialysis for ESRD patients, for CKD patients, for patients with different autoimmune conditions, and for patients in sepsis, AKI, S-AKI, and other life-threatening conditions. It can be used with patients with NFLDS, NASH, peripheral artery disease, Coronary artery disease, and toxic loads of different etiologies.

Given the methods and treatments set forth herein, those of skill in the art are enabled to alter the parameters of apheresis to satisfy patient needs and apparatus requirements. Process metrics such as blood flow, column size, and residence time can vary based on the condition(s) being treated and the number/amount of targets that are being removed or isolated. A common size column for whole blood column will be 40-500 ml, most probably around 100-200 ml. Membrane technology or different high resistance resins can be used as the matrix that is activated with the ligand that targets the compounds to be removed. Plasma separation and cell collection can also be employed, before, during or after the removal of compounds. It is preferable to remove the specific cells prior to the removal of targeted compounds. As is the case with size and number of channels or columns, blood flow may be caried by those of skill in the art based on access and need. If the device/platform employed is a dialysis device, higher volumes of 100-300 ml/minute can be withdrawn, requiring a central line provided with wide enough of tubing/lumen (French #4), double lumen central line catheter, special ports (BARDA and Angiodynamic being two well known brands). Residence time can vary from 30-300 seconds). Flow rate when doing whole blood apheresis requires a high enough flow of blood flow to prevent aggregation of blood cells.

Membrane technology is preferable in whole blood, but high resistance resin can also work. Diameter is usually 3-10 cm based on volume, matrix, and desired blood/plasma flow. This is well known to the skilled artisan.

This application discloses the use of whole blood apheresis as an effective means of treatment of mammalian patients for sepsis and related conditions, as well as various immunotherapy applications. This application also discloses the use of whole blood apheresis for the treatment of mammalian patients and conditions. The ability to treat mammals, including humans, through whole blood apheresis for a wide variety of illnesses and treatments including sepsis and acute kidney injury but certainly not limited thereto, will open the way to treatment through a process that is adaptable to a variety of individuals and situations at a lower cost and less obstacles for a wide variety of conditions. Among the many therapies made more effective, immunotherapies lend themselves to this method.

1. (canceled)

2. (canceled)

3. (canceled)

4. (canceled)

5. (canceled)

6. (canceled)

7. (canceled)

8. A method of treating a mammal with immunotherapy, comprising treating said mammal with plasma exchange to replace plasma of said mammal, followed by apheresis of said mammal so as to selectively withdraw galectin-3.

9. A method of treating a mammal with immunotherapy, comprising treating said mammal with apheresis so as to selectively withdraw galectin-3 from the blood of said mammal, wherein said mammal is subsequently treated with plasma exchange

10. The method of claim 8, wherein said method effectively lowers the level of an agent selected from the group consisting of PD-1, PDL-1, ILIB, IL-6, IL-4, IL-8, TNF Alpha, NF Kappa Beta.

11. The method of claim 10, wherein said agent is PD-1 or PDL-1.

12. The method of claim 8, wherein said method further comprises subjecting the blood of said mammal to hemoperfusion to further treat the blood of said mammal.

13. The method of claim 9, wherein said method comprises subjecting the blood of said mammal to hemoperfusion to further treat the blood of said mammal.

14. The method of claim 8, wherein said plasma exchange comprises total plasma exchange.

15. The method of claim 9, wherein said plasma exchange comprises total plasma exchange,
